SEQUENCE LISTING (19 sequences)

SEQ ID NO: 1 - protein, 41 aa, Homo sapiens
GIGDPVTCLKSGAICHPVFCPRRYKQIGTCGLPGTKCCKKP

SEQ ID NO: 2 - protein, 41 aa, Macaca mulatta
GIGDPVTCLKNGAICHPVFCPRRYKQIGTCGLPGTKCCKKP

SEQ ID NO: 3 - protein, 41 aa, Pan troglodytes
GISDPVTCLKSGAICHPVFCPRRYKQIGTCGLPGTKCCKKP

SEQ ID NO: 4 - protein, 44 aa, Pongo pygmaeus
VFGDISNPVTCLRSGAICHPGFCPRRYKHIGTCGLSVIKCCKKP

SEQ ID NO: 5 - protein, 37 aa, Homo sapiens
  UNSURE (1)...(37): N-terminally truncated hBD2
PVTCLKSGAICHPVFCPRRYKQIGTCGLPGTKCCKKP

SEQ ID NO: 6 - protein, 37 aa, Macaca mulatta
  UNSURE (1)...(37): Macaca mulatta
PVTCLKNGAICHPVFCPRRYKQIGTCGLPGTKCCKKP

SEQ ID NO: 7 - protein, 37 aa, Pan troglodytes; identical to SEQ ID NO: 5
  UNSURE (1)...(37): Pan troglodytes

SEQ ID NO: 8 - protein, 37 aa, Pongo abelii
  UNSURE (1)...(37): N-terminally truncated orangutan BD2
PVTCLRSGAICHPGFCPRRYKHIGTCGLSVIKCCKKP

SEQ ID NO: 9 - protein, 38 aa, Homo sapiens
  UNSURE (1)...(38): Cleaved hBD2 variant (SEQ ID NO 5 + 2 N-terminal AA)
DPVTCLKSGAICHPVFCPRRYKQIGTCGLPGTKCCKKP

SEQ ID NO: 10 - protein, 39 aa, Homo sapiens
  UNSURE (1)...(39): Cleaved hBD2 variant (SEQ ID NO 5 + 2 N-terminal AA)
GDPVTCLKSGAICHPVFCPRRYKQIGTCGLPGTKCCKKP

SEQ ID NO: 11 - protein, 40 aa, Homo sapiens
  UNSURE (1)...(40): Cleaved hBD2 variant (SEQ ID NO 5 + 3 N-terminal AA)
IGDPVTCLKSGAICHPVFCPRRYKQIGTCGLPGTKCCKKP

SEQ ID NO: 12 - protein, 6 aa, Artificial Sequence
  N-terminal variant
IGDPVT

SEQ ID NO: 13 - protein, 6 aa, Artificial Sequence
  N-terminal variant
GDPVTC

SEQ ID NO: 14 - protein, 5 aa, Artificial Sequence
  N-terminal variant
DPVTC

SEQ ID NO: 15 - protein, 5 aa, Artificial Sequence
  N-terminal variant
PVTCL

SEQ ID NO: 16 - protein, 6 aa, Artificial Sequence
  N-terminal variant
PVTCLK

SEQ ID NO: 17 - protein, 31 aa, Artificial Sequence
  Consensus sequence 1
XXGAICHPXFCPRRYKXIGTCGLXXXKCCKP

SEQ ID NO: 18 - protein, 37 aa, Artificial Sequence
  Consensus Sequence 2
PVTCLXXGAICHPXFCPRRYKXIGTCGLXXXKCCKKP

SEQ ID NO: 19 - protein, 4 aa, Artificial Sequence
  truncation
GIGD

(19) United States
(12) Patent Application Publication
(10) Pub. No.: US 2013/0052213 A1
(43) Pub. Date: Feb. 28, 2013

(54) NOVEL IMMUNOMODULATORY PEPTIDE

(71) Applicants: Tanja Maria Rosenkilde Kjaer, Holte (DK); Birgitte Andersen, Bagsvaerd (DK)

(72) Inventors: Tanja Maria Rosenkilde Kjaer, Holte (DK); Birgitte Andersen, Bagsvaerd (DK)

(73) Assignee: Novozymes A/S, Bagsvaerd (DK)

(21) Appl. No.: 13/588,343

(22) Filed: Aug. 17, 2012

Related U.S. Application Data
(60) Provisional application No. 61/559,818, filed on Nov. 15, 2011.

(30) Foreign Application Priority Data
Aug. 19, 2011 (EP) EP 1 117 8133.2

(51) Int. Cl. A61K 39/00; C07K 19/00; C12P 21/02; C12N 15/12; C12N 15/63; C12N 1/21; A61P 19/02; A61P 9/10; A61P 11/00; A61P 17/00; A61P 17/14; A61P 29/00; C07K 14/47; A61P 1/00

(52) U.S. Cl. 424/185.1; 530/324; 435/69.3; 536/23.5; 435/320.1; 435/252.3; 435/252.33

(57) ABSTRACT

The present invention relates a cleaved mammalian beta defensin with immunomodulatory properties. The cleaved or truncated peptides may be as potent as the full length mammalian beta defensins and may be used in the treatment of immunological and autoimmune disorders including but not limited to inflammatory bowel disease, and rheumatoid arthritis.

### RELATED APPLICATIONS

This application claims priority under 35 U.S.C. §119 or 365 to European Application No. EP 1 117 8133.2, filed Aug. 19, 2011, and claims the benefit of U.S. Provisional Application No. 61/559,818, filed Nov. 15, 2011. The entire teachings of the above applications are incorporated herein by reference.

### INCORPORATION BY REFERENCE OF MATERIAL IN ASCII TEXT FILE

This application incorporates by reference the Sequence Listing contained in the following ASCII text file being submitted concurrently herewith:

a) File name: 47681003001seqlist.txt; created Aug. 16, 2012, 9 KB in size.

### FIELD OF THE INVENTION

The present invention relates a cleaved mammalian beta defensin with immunomodulatory properties. The cleaved or truncated peptide can be used in the treatment of immunological disorders including but not limited to inflammatory bowel disease, and rheumatoid arthritis.

### BACKGROUND OF THE INVENTION

Among many other elements, key components of innate immunity are the antimicrobial peptides (AMPs) that individually show considerable selectivity, but collectively are able to rapidly kill a broad spectrum of bacteria, viruses and fungi. The biological significance of AMPs is emphasized by their ubiquitous distribution in nature and they are probably produced by all multicellular organisms. In humans the predominant AMPs are the defensins. The human defensins are small cationic peptides that can be divided into alpha- and beta-defensins based on the topology of their three intramolecular cysteine disulphide bonds. The β-defensins are mainly produced by epithelial cells in various tissues and organs including the skin, trachea, gastrointestinal tract, urogenital system, kidneys, pancreas and mammary gland. The best characterized members of the beta-defensin family are human beta defensins 1-3 (hBD1-3). Some of the human defensins are produced constitutively, whereas others are induced by proinflammatory cytokines or exogenous microbial products.

It has become increasingly clear that the human defensins in addition to their direct antimicrobial activity also have a wide range of immunomodulatory/alternative properties. These include the induction of various chemokines and cytokines, chemotactic and apoptotic activities, induction of prostaglandin, histamine and leukotriene release, inhibition of complement, stimulation of dendritic cell maturation through toll-like receptor signalling and stimulation of pathogen clearance by neutrophils. Furthermore, the human defensins also play a role in wound healing, proliferation of epithelial and fibroblast cells, angiogenesis and vasculogenesis.

WO 2007/081486 suggests the use of human defensins in the treatment of inflammatory bowel disease. The inventors suggest that defensins administered orally to Crohn's patients, in a formulation that allow their release at proper locations in the intestinal lumen, would reduce the number of invading bacteria, re-establish a normal epithelial barrier function and, thus, reduce the severity of the inflammatory disease.

WO 2010/007166 discloses treatment of inflammatory bowel diseases with mammalian beta defensins, in particular human beta defensin 2. WO 2010/007168 discloses treatment of rheumatoid arthritis using mammalian beta defensins, in particular human beta defensin 2. WO 2010/007165 discloses treatment of immunological disorders with mammalian defensions, in particular with human beta-defensin 2 (hBD2).

### SUMMARY OF THE INVENTION

The present invention demonstrates an anti-inflammatory activity of cleaved hBD2 in an in vitro model using human peripheral blood mononuclear cells (PBMC). The term “cleaved defensin” means that the peptide has been cleaved compared to the normal wildtype peptide by removing one or more terminal amino acids. In other words, a cleaved peptide is shorter than the peptide from which it is cleaved. The process of removing terminal amino acids from a polypeptide or a peptide may also be referred to as truncation. Accordingly, a “cleaved defensin” is also a “truncated defensin”. The cleaved and truncated defensins of the invention are N-terminally truncated or cleaved by having a shorter N-terminal compared to the wildtype sequences.

This does not necessarily mean that the product has to be the result of cleavage of a longer product. Having realized that shorter versions of wildtype mammalian defensins are bioactive, it is conceivable to manufacture these directly without the need for actual cleavage.

Preferably the cleaved or truncated mammalian defensin is a cleaved or truncated mammalian beta-defensin 2.

The activity of a representative cleaved or truncated mammalian beta-defensin 2, hBD2, surprisingly is the same as previously reported for full-length hBD2 indicating that also the cleaved hBD2 would have a beneficial treatment effect on IBD and other disorders that can be treated with hBD2. This is particularly unexpected as the sequence deleted from the full length hBD2 is very strongly conserved among species. The strong conservation normally indicates that the conserved residues are important for the function of the peptide.

Compared to the full length mammalian beta defensin, the cleaved beta defensins of the present invention are shorter, and therefore smaller molecules. As the molecules are shorter an effective dosage giving the same concentration of peptide is smaller in mg or μg than for full-length mammalian beta-defensin.

The cleaved mammalian beta-defensins of the invention are unexpectedly stable for extended time and at high temperature in a strongly acidic environment. The cleaved mammalian beta-defensins are at least as potent as full length mammalian beta-defensins.

More specifically, the invention relates to a peptide selected from the group consisting of
a) a peptide having the amino acid sequence of any of SEQ ID NO: 5, 6, 7, 8, 9, 10, or 11;
b) an anti-inflammatory peptide variant of the peptide of a) having 1-5 amino acid substitutions compared to the SEQ ID NOs;
c) an anti-inflammatory peptide variant having at least 80% sequence identity to any of SEQ ID NO: 5, 6, 7, and 8 and having an N-terminal selected from the group consisting of IGDPVT- (SEQ ID NO:12), GDPVTC- (SEQ ID NO:13), DPVTCL- (SEQ ID NO:14) and PVTCL-(SEQ ID NO:15), preferably PVTCL- (SEQ ID NO:15); and
d) an anti-inflammatory peptide having the consensus sequence: P V T C L X₁ X₂ G A I C H P X₃ F C P R R Y K X₄ I G T C G L X₅ X₆ X₇ K C C K K P (SEQ ID NO:18), wherein independently
   X₁ is K or R,
   X₂ is S or N,
   X₃ is V or G,
   X₄ is Q or H,
   X₅ is P or 5,
   X₆ is G or V, and
   X₇ is T or I.

In another aspect the invention relates to a method of manufacturing the peptide of any of the invention, said method comprising recombinant expression, the use of an automated amino acid synthesiser, or acid cleavage of a full length mammalian beta-defensin 2.

In a further aspect, the invention relates to a polynucleotide comprising a nucleotide sequence selected from the group consisting of:
a) a polynucleotide coding for a polypeptide consisting of an N-terminal eukaryotic signal sequence linked to a peptide the invention; and
b) a polynucleotide coding for a polypeptide consisting of an N-terminal methionine (M) linked to a cleavable linker, and a peptide of the invention.

The invention furthermore relates to an expression vector comprising the polynucleotide of the invention and to a cell being transduced or transfected with the expression vector of the invention.

In a still further aspect, the invention relates to a pharmaceutical composition comprising the peptide of the invention, and a pharmaceutical acceptable excipient, diluent or carrier.

The invention furthermore relates to the peptide of the invention for use as a medicament, in particular in the treatment of autoimmune disorders and inflammatory disorders and to methods of treatment of these disorders.

### BRIEF DESCRIPTION OF THE DRAWINGS

The foregoing will be apparent from the following more particular description of example embodiments of the invention, as illustrated in the accompanying drawings in which like reference characters refer to the same parts throughout the different views. The drawings are not necessarily to scale, emphasis instead being placed upon illustrating embodiments of the present invention.

FIG. 1. IL-10 production from peripheral blood mononuclear cell (PBMCs treated with either full-length or cleaved hBD2 for comparison. Both full-length hBD2 and cleaved hBD2 significantly induced IL-10 production in PBMCs stimulated with LPS. Dexamehtasone and Infliximab are used as controls for anti-inflammatory effects (down-regulation of TNF), but neither Infliximab (anti-TNF antibody) nor Dexamethasone up-regulate IL-10, on the contrary they down-regulate IL-10, which we have observed for many donors.

FIG. 2. TNF production from PBMCs treated with either full-length or cleaved hBD2 for comparison. Both full-length HBD2 and cleaved hBD2 significantly reduced TNF production in PBMCs stimulated with LPS. Dexamehtasone and Infliximab are used as controls for anti-inflammatory effects (down-regulation of TNF).

FIG. 3. Clustal W (2.1) multiple sequence alignment of human (SEQ ID NO:1), Rhesus macaque (SEQ ID NO:2), chimpanzee (SEQ ID NO:3) and orangutan (SEQ ID NO:4) beta defensin 2.

FIG. 4. Clustal W (2.1) multiple sequence alignment of preferred cleaved human (SEQ ID NO:5), Rhesus macaque (SEQ ID NO:6), chimpanzee (SEQ ID NO:7) and orangutan (SEQ ID NO:8) beta defensin 2.

* indicates positions which have a single, fully conserved residue.
: indicates that one of the following ‘strong’ groups is fully conserved:
S,T,A; N,E,Q,K; N,H,Q,K; N,D,E,Q; Q,H,R,K; M,I,L,V; M,I,L,F; H,Y; F,Y,W.
   . indicates that one of the following ‘weaker’ groups is fully conserved:
C,S,A; A,T,V; S,A,G; S,T,N,K; S,T,P,A; S,G,N,D; S,N,D,E,Q,K; N,D,E,Q,H,K; N,E,Q,H,R,K; V,L,I,M; H,F,Y.

### DETAILED DESCRIPTION OF THE INVENTION

### Definitions

Autoimmune disease: The term “autoimmune disease” as used herein refers to a disease arising from an overactive immune response of the body against substances and tissues normally present in the body. The terms autoimmune disease and autoimmune disorder may be used interchangeable in the present application.

Defensin: The term “defensin” as used herein refers to polypeptides recognized by a person skilled in the art as belonging to the defensin class of antimicrobial peptides. To determine if a polypeptide is a defensin according to the invention, the amino acid sequence may be compared with the hidden markov model profiles (HMM profiles) of the PFAM database by using the freely available HMMER software package.

The PFAM defensin families include for example Defensin_{—}1 or “Mammalian defensin” (accession no. PF00323), and Defensin_{—}2 or Defensin beta or “Beta Defensin” (accession no. PF00711).

The defensins of the invention belong to the beta defensin class. The defensins from the beta defensin class share common structural features known to the person skilled in the art, such as the cysteine pattern.

More specifically, the cleaved mammalian beta-defensins of the invention are cleaved mammalian beta-defensin 2.

Identity: The relatedness between two amino acid sequences or between two nucleotide sequences is described by the parameter “identity”.

For purposes of the present invention, the degree of identity between two amino acid sequences is determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends in Genetics 16: 276-277; //emboss.org), preferably version 3.0.0 or later. The optional parameters used are gap open penalty of 10, gap extension penalty of 0.5, and the EBLOSUM62 (EMBOSS version of BLOSUM62) substitution matrix. The output of Needle labeled “longest identity” (obtained using the—nobrief option) is used as the percent identity and is calculated as follows:

(Identical Residues×100)/(Length of Alignment−Total Number of Gaps in Alignment)

Isolated peptide: The term “isolated peptide” as used herein refers to a peptide that is isolated from a source. In one aspect, the peptide is at least 1% pure, preferably at least 5% pure, more preferably at least 10% pure, more preferably at least 20% pure, more preferably at least 40% pure, more preferably at least 60% pure, even more preferably at least 80% pure, and most preferably at least 90% pure, as determined by RP-HPLC analysis.

Substantially pure peptide: The term “substantially pure peptide” denotes herein a peptide preparation that contains at most 10%, preferably at most 8%, more preferably at most 6%, more preferably at most 5%, more preferably at most 4%, more preferably at most 3%, even more preferably at most 2%, most preferably at most 1%, and even most preferably at most 0.5% by weight of other peptide material with which it is natively or recombinantly associated. It is, therefore, preferred that the substantially pure peptide is at least 92% pure, preferably at least 94% pure, more preferably at least 95% pure, more preferably at least 96% pure, more preferably at least 96% pure, more preferably at least 97% pure, more preferably at least 98% pure, even more preferably at least 99%, most preferably at least 99.5% pure by weight of the total peptide material present in the preparation. The peptides of the present invention are preferably in a substantially pure form. This can be accomplished, for example, by preparing the peptide by well-known recombinant methods or by classical purification methods.

Inflammatory bowel disease (IBD): inflammatory bowel disease (IBD) is a group of inflammatory conditions of the colon and small intestine. The major types of IBD are Crohn's disease and ulcerative colitis (UC).

Accounting for far fewer cases are other forms of IBD, which are not always classified as typical IBD:
Collagenous colitis
Lymphocytic colitis
Ischaemic colitis
Diversion colitis
Behcet's disease
Indeterminate colitis

The main difference between Crohn's disease and Ulcerative colitis (UC) is the location and nature of the inflammatory changes. Crohn's can affect any part of the gastrointestinal tract, from mouth to anus (skip lesions), although a majority of the cases start in the terminal ileum. Ulcerative colitis, in contrast, is restricted to the colon and the rectum.

Treatment: The terms “treatment” and “treating” as used herein refer to the management and care of a patient for the purpose of combating a condition, disease or disorder. The term is intended to include the full spectrum of treatments for a given condition from which the patient is suffering, such as administration of the active compound for the purpose of: alleviating or relieving symptoms or complications; delaying the progression of the condition, disease or disorder; curing or eliminating the condition, disease or disorder; and/or preventing the condition, disease or disorder, wherein “preventing” or “prevention” is to be understood to refer to the management and care of a patient for the purpose of hindering, reducing or delaying the development of the condition, disease or disorder, and includes the administration of the active compounds to prevent or reduce the risk of the onset of symptoms or complications. The patient to be treated is preferably a mammal, in particular a human being. The patients to be treated according to the present invention can be of various ages.

### Mammalian Beta Defensins

The present invention relates to truncated or cleaved mammalian beta defensins, such as monkey or human beta defensins, more preferably Homimidae beta defensins, more preferably human beta defensin. In a particularly preferred embodiment of the invention the cleaved or truncated mammalian beta-defensin is of type 2.

The cleaved mammalian beta-defensins of the invention are unexpectedly stable for extended time and at high temperature in a strongly acidic environment. In particular, cleaved mammalian beta-defensins with the N-terminal PVTCL- (SEQ ID NO:15) are extremely stable at high temperature and low pH.

In a preferred embodiment, the peptide of the invention is selected from the group consisting of peptides having the sequence of SEQ ID NO:5, 6, 7, and 8, preferably SEQ ID NO:5.

Variants of these sequences may have 1-5 amino acid modifications, preferably 1-4 amino acid modifications, more preferably 1-3 amino acid modifications, most preferably 1-2 amino acid modification(s) compared to one of SEQ ID NO:5, 6, 7, and 8, most preferably compared to SEQ ID NO:5.

In an embodiment of the present invention, the N-terminal of the cleaved mammalian beta defensins comprises a sequence selected from the group consisting of: IGDPVT- (SEQ ID NO:12), GDPVTC- (SEQ ID NO:13), DPVTCL- (SEQ ID NO:14) and PVTCL- (SEQ ID NO:15), preferably PVTCL- (SEQ ID NO:15).

In an embodiment, the cleaved mammal beta defensins of the present invention is at the most 40 amino acids long, preferably 39 amino acids long, such as 38 amino acids, more preferably 37 amino acids long.

Still more preferably, the peptide is exactly 37 amino acids long. Even more preferably, this peptide being 37 amino acids long has the N-terminal PVTCL-. More specifically, the 37 amino acid long peptide may have up to 5 amino acid substitutions compared to SEQ ID NO:5, more preferably up to 4, more preferably up to 3, more preferably up to 2, more preferably up to 1 amino acid substitution.

In one embodiment of the invention, the peptides do not comprise an N-terminal GIGD (SEQ ID NO:19) sequence. More preferably the peptides do not comprise any GIGD (SEQ ID NO:19) sequence anywhere.

In an embodiment, the cleaved mammalian beta defensins of the invention have a degree of identity of at least 80%, preferably at least 85%, more preferably at least 90%, and most preferably at least 95% to any of the amino acid sequences of SEQ ID NO:5, SEQ ID NO:6, SEQ ID NO:7, or SEQ ID NO:8.

In a preferred embodiment, the mammalian beta defensins of the invention have a degree of identity of at least 80%, preferably at least 85%, more preferably at least 90%, and most preferably at least 95% to the amino acid sequence of SEQ ID NO:5. In a preferred embodiment, the mammalian beta defensins of the invention consist of cleaved human beta defensin 2 (SEQ ID NO:5).

In a preferred embodiment of the invention, the mammalian beta defensin is selected from the group consisting of PVTCLKSGAICHPVFCPRRYKQIGTCGLPGTKCCKKP (SEQ ID NO:5), DPVTCLKSGAICHPVFCPRRYKQIGTCGLPGTKCCKKP (SEQ ID NO:9), GDPVTCLKSGAICHPVFCPRRYKQIGTCGLPGTKCCKKP (SEQ ID NO:10), and IGDPVTCLKSGAICHPVFCPRRYKQIGTCGLPGTKCCKKP (SEQ ID NO:11); and variants of these sequences having 1-5 amino acid modifications, preferably 1-4 amino acid modifications, more preferably 1-3 amino acid modifications, most preferably 1-2 amino acid modification(s) compared to one of SEQ ID NO:5, 9, 10, and 11).

More preferably the cleaved peptides of the invention comprise 37 contiguous residues (SEQ ID NO:5) from human beta defensin 2 with up to 5 amino acid modifications, preferably up to 4, more preferably up to 3, more preferably up to 2, more preferably 1 amino acid modification, and no further human beta-defensin sequence. Accordingly the peptides of the invention may comprise additional amino acids from other proteins or peptides, for example in the shape of tags or fusion proteins as long as these additional amino acids are not found at corresponding positions in human beta-defensin 2.

In a particularly preferred embodiment the cleaved mammalian beta-defensin of the invention is 37 amino acids long, has an N-terminal with the following sequence PVTCLK (SEQ ID NO:16)-, and has 1-5 amino acid modifications, preferably 1-4 amino acid modifications, more preferably 1-3 amino acid modifications, most preferably 1-2 amino acid modification(s) compared to SEQ ID NO:5.

The cleaved mammalian beta defensin of the present invention may also be described by the following consensus sequence:

(SEQ ID NO: 18)
P V T C L X₁ X₂ G A I C H P X₃ F C P R R Y K X₄ I
G T C G L X₅ X₆ X₇ K C C K K P

Wherein independently;

### X₁ is K or R,

### X₂ is S or N,

### X₃ is V or G,

### X₄ is Q or H,

### X₅ is P or S,

### X₆ is G or V, and

### X₇ is T or I.

Preferably the peptides of the invention are provided in isolated and/or substantially pure form.

The bioactive peptide is preferably in the form of a dimer, preferably a homo-dimer.

### Functional Equivalent Variant

In the context of the present invention, a “functionally equivalent variant” of a cleaved mammalian (e.g. human) beta defensin is a modified mammal (e.g. human) beta defensin exhibiting approx. the same effect on the activity of pro-inflammatory cytokines and chemokines and/or anti-inflammatory cytokine as the full-length mammalian (e.g. human) beta defensin in in vitro and in vivo assays such as the assay of example 2. By approximately the same effect is intended an effect of the peptide of the invention on pro-inflammatory cytokines or chemokines and/or anti-inflammatory cytokines which effect is at least 50% of the effect of hBD2 (SEQ ID NO:1) in the same assay at the same concentration (μg/mL), more preferably at least 60%, more preferably at least 70%, more preferably at least 75%, more preferably at least 80%, more preferably at least 90%, more preferably wherein the peptide has an effect which is not significantly lower than the activity of hBD2 (SEQ ID NO:1). Still more preferably, the peptides of the invention are capable of inducing anti-inflammatory cytokines and reducing pro-inflammatory chemokines and cytokines to approximately the same extent as hBD2 at the same concentration. In some embodiments of the invention, the peptides exhibit higher activity than the wildtype (full length) peptide.

In one embodiment the functionally equivalent variant exhibits an improved effect on the activity of pro-inflammatory cytokines and chemokines and/or anti-inflammatory cytokine compared to the full-length mammal (e.g. human) beta defensin.

In one embodiment the effect on the activity of pro-inflammatory and anti-inflammatory cytokines is measured as described in WO 2010/007166 in Example 4 and 6. The effect may be measured in a human cell selected from the group consisting of human PBMCs, human CD14+ monocyte-derived dendritic cells, a human monocyte cell line, and human immature dendritic cells, preferably human PBMC.

The activity of the peptides of the invention is preferably a down-regulation of the activity of at least one cytokine selected from the group consisting of the pro-inflammatory cytokines and chemokines. Preferably the activity is a down-regulation of the activity of at least one cytokine selected from the group consisting of the pro-inflammatory IL-23, IL-1β, IL-6, IL-8, MCP-1, and TNFα, and/or an up-regulation of the activity of the anti-inflammatory cytokine IL-10.

According to the invention, a functionally equivalent variant of a cleaved mammalian (e.g. human) beta defensin may comprise 1-5 amino acid modifications, preferably 1-4 amino acid modifications, more preferably 1-3 amino acid modifications, most preferably 1-2 amino acid modification(s), and in particular one amino acid modification, as compared to the cleaved mammal (e.g. human) beta defensin amino acid sequence. Preferably compared to cleaved human beta defensin 2, having SEQ ID NO:5.

The term “modification” means herein any chemical modification of a cleaved mammal (e.g. human) beta defensin. The modification(s) can be substitution(s), deletion(s) and/or insertions(s) of the amino acid(s) as well as replacement(s) of amino acid side chain(s); or use of unnatural amino acids with similar characteristics in the amino acid sequence. In particular the modification(s) can be amidations, such as amidation of the C-terminus. Preferably, the modification is an amino acid substitution, more preferably a conservative or semi-conservative substitution as defined by the Clustal W groups.

Preferably, amino acid modifications are of a minor nature, that is conservative amino acid substitutions or insertions that do not significantly affect the folding and/or activity of the polypeptide; single deletions; small amino- or carboxyl-terminal extensions, which are not beta-defensin derived.

In one embodiment the cleaved peptides comprise a small extension that facilitates purification by changing net charge or another function, such as a poly-histidine tag, an antigenic epitope or a binding domain. In one embodiment the small extension, such as a poly-histidine tag, an antigenic epitope or a binding domain is attached to the cleaved mammalian (e.g. human) beta defensin through a small linker peptide of up to about 20-25 residues and said linker may contain an enzyme cleavage site or an alternative cleavage site, such as a site that can be cleaved by acid. In another embodiment a small tag may be added to the N-terminal or the C-terminal of the peptide. This tag preferably is not a mammalian beta-defensin 2 or a fragment thereof.

An N-terminal extension of the polypeptides of the invention may suitably consist of from 1 to 50 amino acids, preferably 2-20 amino acids, especially 3-15 amino acids. In one embodiment N-terminal peptide extension does not contain an Arg (R). In another embodiment the N-terminal extension comprises a kex2 or kex2-like cleavage site as will be defined further below. In a preferred embodiment the N-terminal extension is a peptide, comprising at least two Glu (E) and/or Asp (D) amino acid residues, such as an N-terminal extension comprising one of the following sequences: EAE, EE, DE and DD. The extension preferably is not a mammalian beta-defensin 2 or a fragment thereof.

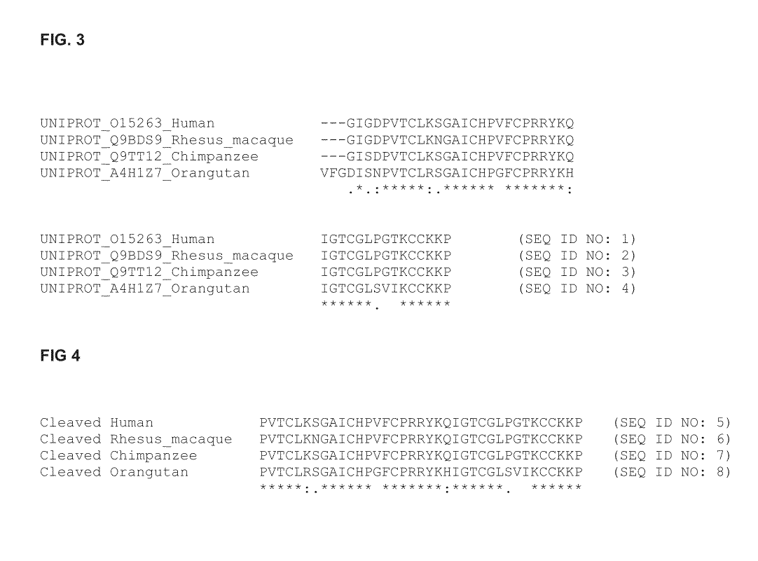

The Clustal W alignments in FIG. 3 or FIG. 4 can be used to predict which amino acid residues can be substituted without substantially affecting the biological activity of the protein. The sequences were aligned using Clustal W 2.1 (www.genome.jp/tools/clustalw/) and the following settings: Gap Open Penalty: 10, Gap Extension Penalty: 0.05, Weight Transition: NO, Hydrophilic Residues for Proteins: GPSNDQE, Hydrophilic Gaps: YES,

### Weight Matrix: BLOSUM (for PROTEIN).

Substitutions within the following group (Clustal W, ‘strong’ conservation group) are to be regarded as conservative substitutions within the meaning of the present invention
S,T,A; N,E,Q,K; N,H,Q,K; N,D,E,Q; Q,H,R,K; M,I,L,V; M,I,L,F; H,Y; F,Y,W.

Substitutions within the following group (Clustal W, ‘weak’ conservation group) are to be regarded as semi-conservative substitutions within the meaning of the present invention
C,S,A; A,T,V; S,A,G; S,T,N,K; S,T,P,A; S,G,N,D; S,N,D,E,Q,K; N,D,E,Q,H,K; N,E,Q,H,R,K; V,L,I,M; H,F,Y.

Examples of conservative substitutions are substitutions made within the group of basic amino acids (arginine, lysine and histidine), acidic amino acids (glutamic acid and aspartic acid), polar amino acids (glutamine and asparagine), hydrophobic amino acids (leucine, isoleucine and valine), aromatic amino acids (phenylalanine, tryptophan and tyrosine), and small amino acids (glycine, alanine, serine, threonine and methionine). Amino acid substitutions which do not generally alter specific activity are known in the art and are described, for example, by H. Neurath and R. L. Hill, 1979, In, The Proteins, Academic Press, New York. The most commonly occurring exchanges are Ala/Ser, Val/Ile, Asp/Glu, Thr/Ser, Ala/Gly, Ala/Thr, Ser/Asn, Ala/Val, Ser/Gly, Tyr/Phe, Ala/Pro, Lys/Arg, Asp/Asn, Leu/Ile, Leu/Val, Ala/Glu, and Asp/Gly.

In addition to the 20 standard amino acids, non-standard amino acids (such as 4-hydroxyproline, 6-N-methyl lysine, 2-aminoisobutyric acid, isovaline, and alpha-methyl serine) may be substituted for amino acid residues of a wild-type polypeptide. A limited number of non-conservative amino acids, amino acids that are not encoded by the genetic code, and unnatural amino acids may be substituted for amino acid residues. “Unnatural amino acids” have been modified after protein synthesis, and/or have a chemical structure in their side chain(s) different from that of the standard amino acids. Unnatural amino acids can be chemically synthesized, and preferably, are commercially available, and include pipecolic acid, thiazolidine carboxylic acid, dehydroproline, 3- and 4-methylproline, and 3,3-dimethylproline.

Essential amino acids in a mammal beta defensin can be identified according to procedures known in the art, such as site-directed mutagenesis or alanine-scanning mutagenesis (Cunningham and Wells, 1989, Science 244: 1081-1085). In the latter technique, single alanine mutations are introduced at every residue in the molecule, and the resultant mutant molecules are tested for biological activity (i.e., activity against an inflammatory bowel disease and/or suppresion of TNF-alpha activity) to identify amino acid residues that are critical to the activity of the molecule. See also, Hilton et al., 1996, J. Biol. Chem. 271: 4699-4708. The identities of essential amino acids can also be inferred from analysis of identities with polypeptides which are related to mammal beta defensins (see Clustal W alignment in FIG. 4 and FIG. 5).

Single or multiple amino acid substitutions can be made and tested using known methods of mutagenesis, recombination, and/or shuffling, followed by a relevant screening procedure, such as those disclosed by Reidhaar-Olson and Sauer, 1988, Science 241: 53-57; Bowie and Sauer, 1989, Proc. Natl. Acad. Sci. USA 86: 2152-2156; WO 95/17413; or WO 95/22625. Other methods that can be used include error-prone PCR, phage display (e.g., Lowman et al., 1991, Biochem. 30:10832-10837; U.S. Pat. No. 5,223,409; WO 92/06204), and region-directed mutagenesis (Derbyshire et al., 1986, Gene 46:145; Ner et al., 1988, DNA 7:127).

When the result of a given substitution cannot be predicted with certainty, the derivatives may be readily assayed according to the methods described herein above to determine the presence or absence of biological activity.

The peptides of the invention may also be modified by covalently linking the peptides to another chemical moiety such as a different peptide or polypeptide, an affinity tag, a carbohydrate, a lipid, and a synthetic polymer.

### Methods and Uses

The present invention demonstrates an anti-inflammatory activity of cleaved hBD2 in an in vitro model using human peripheral blood mononuclear cells (PBMC). The observed activity is at least the same as previously reported for full-length hBD2 indicating that also the cleaved hBD2 would have a beneficial effect in the treatment of autoimmune diseases such as IBD. As shown in FIG. 2, it appears that cleaved human beta-defensin 2 (SEQ ID NO:5) at some concentrations are more potent than full length human beta-defensin 2 (SEQ ID NO:1).

Cleaved hBD2 thus shows potential as a medicament for treatment of inflammatory bowel diseases, such as ulcerative colitis and Crohn's disease.

A well-recognized model for studying IBD is the DSS colitis mouse model, as described in Kawada et al. “Insights from advances in research of chemically induced experimental models of human inflammatory bowel disease”, World J. Gastroenterol., Vol. 13 (42), pp. 5581-5593 (2007); and Wirtz and Neurath “Mouse models of inflammatory bowel disease”, Advanced Drug Delivery Reviews, Vol. 59 (11), 1073-1083 (2007).

Inflammatory bowel disease according to this invention relates to Crohn's Disease and ulcerative colitis.

In yet another embodiment of the present invention administration of a cleaved mammalian beta defensin may be used for modulating cytokine production at the epithelial lining in the gastrointestinal tract or systemically through the interaction at the epithelial lining.

The modulation is preferably a down-regulation of the activity of at least one cytokine selected from the group consisting of the pro-inflammatory cytokines and chemokines. Preferably the modulation is a down-regulation of the activity of at least one cytokine selected from the group consisting of the pro-inflammatory IL-23, IL-1β and TNFα, and/or an up-regulation of the activity of the anti-inflammatory cytokine IL-10.

In another embodiment the present invention provides methods of treating inflammatory bowel diseases, which treatment comprises administration of an effective amount of a cleaved mammalian beta defensin in the form of a pharmaceutical composition.

In another embodiment the present invention provides methods of treating rheumatoid arthritis, which treatment comprises administration of an effective amount of a cleaved mammalian beta defensin in the form of a pharmaceutical composition. Full length hBD2 (SEQ ID NO:1) has shown therapeutic potential in an animal model of rheumatoid arthritis (WO 2010/007168).

It is also conceivable that cleaved mammalian beta-defensins of this invention are useful for treating an inflammatory disease or disorder selected from the group consisting of rheumatoid arthritis, psoriasis, osteoarthritis, multiple sclerosis, artherosclerosis, scleroderma (systemic sclerosis), lupus, systemic lupus erythematosus (SLE), (acute) glomerulonephritis, asthma, chronic obstructive pulmonary diseases (COPD), respiratory distress-syndrome (ARDS), vasculitis, uveitis, dermatitis, atopic dermatitis, alopecia, rhinitis (allergica), allergic conjunctivitis, myasthenia gravis, sclerodermitis, sarcoidosis, psoriatic arthritis, ankylosing spondylitis, juvenile idiopathic arthritis, Graves disease, Sjogren's syndrome, and Behget disease. These are indications for which full length human beta-defensin 2 has shown potential (WO 2010/007165).

### Administration Routes

Cleaved mammalian beta defensins can be employed therapeutically in compositions formulated for administration by any conventional route. In one embodiment of the present invention cleaved mammalian beta defensins are administered orally. This is possible because the cleaved mammalian beta-defensins of the invention have a very high degree of acid stability. The peptides are resistant against acid hydrolysis for 16 hrs and 85° C. at pH 2.3. A particularly acid stable peptide of the invention has the N-terminal: PVTCL- (SEQ ID NO:15).

In another embodiment of the present invention cleaved mammalian beta defensins are administered by parenteral administration.

In another embodiment of the present invention cleaved mammalian beta defensins are administered by systemic administration.

Oral administration is normally for enteral drug delivery, wherein the agent is delivered through the enteral mucosa.

Parenteral administration is any administration route not being the oral/enteral route whereby the medicament avoids first-pass degradation in the liver. Accordingly, parenteral administration includes any injections and infusions, for example bolus injection or continuous infusion, such as intramuscular administration and subcutaneous administration. The subcutaneous and intramuscular forms of parenteral administration are generally preferred.

Systemic administration is normally administration directly into the bloodstream of the subject to be treated. This may in one embodiment be i.v. injection.

Within yet other embodiments, compositions, of preferred embodiments may be formulized as a lyophilizate, utilizing appropriate excipients that provide stability as a lyophilizate, and subsequent to rehydration.

Pharmaceutical compositions containing a cleaved mammal beta defensin, such as a cleaved human beta defensin, can be manufactured according to conventional methods, e.g., by mixing, granulating, coating, dissolving or lyophilizing processes.

Pharmaceutical compositions of preferred embodiments comprise a cleaved mammal beta defensin, such as a cleaved human beta defensin, and a pharmaceutically acceptable carrier, excipient and/or diluent.

Pharmaceutically acceptable carriers and/or diluents are familiar to those skilled in the art. For compositions formulated as liquid solutions, acceptable carriers and/or diluents include saline and sterile water, and may optionally include antioxidants, buffers, bacteriostats, and other common additives.

Parenteral administration may be in the form of injections and infusions and said formulations may take such forms as suspensions, solutions, or emulsions in oily or aqueous vehicles. Alternatively, the active ingredient may be in powder form, obtained by aseptic isolation of sterile solid or by lyophilisation from solution for constitution before use with a suitable vehicle, e.g., sterile, pyrogen-free water. The formulations can be presented in unit-dose or multi-dose sealed containers, such as ampoules, vials, pre-filled syringes, infusion bags, or can be stored in a freeze-dried (lyophilized) condition requiring only the addition of the sterile liquid excipient, for example, water, for injections, immediately prior to use.

Examples of oily or non-aqueous carriers, diluents, solvents or vehicles include propylene glycol, polyethylene glycol, vegetable oils, and injectable organic esters, and may contain formulation agents such as preserving, wetting, emulsifying or suspending, stabilizing and/or dispersing agents.

The compound of the present invention may be formulated in a wide variety of formulations for oral administration. Solid form preparations may include powders, tablets, drops, capsules, cachets, lozenges, and dispersible granules. Other forms suitable for oral administration may include liquid form preparations including emulsions, syrups, elixirs, aqueous solutions, aqueous suspensions, toothpaste, gel dentrifrice, chewing gum, or solid form preparations which are intended to be converted shortly before use to liquid form preparations, such as solutions, suspensions, and emulsions.

In powders, the carrier is a finely divided solid which is a mixture with the finely divided active component. In tablets, the active component is mixed with the carrier having the necessary binding capacity in suitable proportions and compacted in the shape and size desired. Suitable carriers are magnesium carbonate, magnesium stearate, talc, sugar, lactose, pectin, dextrin, starch, gelatin, tragacanth, methylcellulose, sodium carboxymethylcellulose, a low melting wax, cocoa butter, and the like.

Drops according to the present invention may comprise sterile or non-sterile aqueous or oil solutions or suspensions, and may be prepared by dissolving the active ingredient in a suitable aqueous solution, optionally including a bactericidal and/or fungicidal agent and/or any other suitable preservative, and optionally including a surface active agent. Suitable solvents for the preparation of an oily solution include glycerol, diluted alcohol and propylene glycol.

Emulsions may be prepared in solutions in aqueous propylene glycol solutions or may contain emulsifying agents such as lecithin, sorbitan monooleate, or acacia. Aqueous solutions can be prepared by dissolving the active component in water and adding suitable colorants, flavors, stabilizing and thickening agents. Aqueous suspensions can be prepared by dispersing the finely divided active component in water with viscous material, such as natural or synthetic gums, resins, methylcellulose, sodium carboxymethylcellulose, and other well-known suspending agents.

The formulation can contain (in addition to a cleaved mammal beta defensin, and other optional active ingredients) carriers, fillers, disintegrators, flow conditioners, sugars and sweeteners, fragrances, preservatives, stabilizers, wetting agents, emulsifiers, solubilizers, salts for regulating osmotic pressure, buffers, diluents, dispersing and surface-active agents, binders, lubricants, and/or other pharmaceutical excipients as are known in the art.

One skilled in this art may further formulate cleaved mammal beta defensins in an appropriate manner, and in accordance with accepted practices, such as those described in Remington's Pharmaceutical Sciences, Gennaro, Ed., Mack Publishing Co., Easton, Pa. 1990.

A cleaved mammal beta defensin, such as a cleaved human beta defensin, can be used alone, or in combination therapies with one, two, or more other pharmaceutical compounds or drug substances, and/or with one or more pharmaceutically acceptable excipient(s).

### Methods of Manufacture

The cleaved mammal beta defensins may be prepared by in vitro synthesis, using conventional methods as known in the art. Various commercial synthetic apparatuses are available, for example automated synthesizers by Applied Biosystems Inc., Beckman, etc. By using synthesizers, naturally occurring amino acids may be substituted with unnatural amino acids, particularly D-isomers (or D-forms) e.g. D-alanine and D-isoleucine, diastereoisomers, side chains having different lengths or functionalities, and the like. The particular sequence and the manner of preparation will be determined by convenience, economics, purity required, and the like.

The cleaved mammal beta defensins may also be isolated and purified in accordance with conventional methods of recombinant synthesis in both prokaryotic and eukaryotic hosts in which the expression cassette encodes the truncated form. The cleaved mammal beta defensins may be produced by the expression host and the defensins purified using liquid chromatography methods as RP-HPLC, ion exchange chromatography, size exclusion chromatography, affinity chromatography, or other purification techniques as crystallization, precipitation and filtration techniques.

For expression in eukaryotic host cells, the sequence coding for the cleaved mammalian beta-defensin of the invention is fused to a sequence coding for an appropriate signal sequence to ensure secretion of the peptide into the surrounding medium.

For expression in bacterial host cells, the coding sequence may be fused to an appropriate tag for purification, the sequence comprising a start methionine. Preferably the tag comprises an cleavage site such as an enterokinase cleavage site, allowing subsequent removal of the tag. In a preferred embodiment the cleavage site is an acid-cleavage site. Such sites can be cleaved merely by the use of acid thus obviating the need for expensive enzymes. This is also possible due to the high stability of the peptides of the invention against acid hydrolysis even at elevated temperatures.

The cleaved mammal beta defensins may also be produced by acid hydrolysis of the D-P (aspartic acid-proline) peptide bond in partly or fully purified mammal beta defensins. The acid hydrolysis at elevated temperatures may be employed as an additional step in a standard purification process for mammal beta defensins followed by the remainder purification steps. Alternatively the acid hydrolysis can be employed at the final purified full length mammal beta defensins and the cleaved amino acids removed by an additional purification step using liquid chromatography methods as RP-HPLC, ion exchange chromatography, size exclusion chromatography, affinity chromatography, or other purification techniques as crystallization, precipitation and filtration techniques. The condition for the acid hydrolysis is optimized with respect to pH, temperature and time for each product solution as parameters as defensin content and purity affect the hydrolysis rate.

### Polynucleotides

The peptide of the invention may be expressed from polynucleotides inserted into appropriate expression vectors using methods know in the art. Suitable a polynucleotide comprise a nucleotide sequence selected from the group consisting of:
a) a polynucleotide coding for a polypeptide consisting of an N-terminal eukaryotic signal sequence linked to a peptide the invention; and
b) a polynucleotide coding for a polypeptide consisting of an N-terminal methionine (M) linked to a cleavable linker, and a peptide of the invention.

The first group of polynucleotides may be used for expression in eukaryotic host cells which are capable of secreting the peptides into the medium when the coding sequence is equipped with a suitable signal sequence. The selection of suitable signal sequences for appropriate expression hosts, fungal, yeast, insect, mammalian and other cells is known in the art. It is possible to predict correct processing of signal sequences using available prediction algorithms such as SignalP.

The second group of polynucleotides can be used in prokaryotic expression systems. The N-terminal Methionine and the cleavable linker may be removed by enzymatic or chemical cleavage. Preferably the cleavage site is an acid-cleavage site which can be cleaved by inexpensive acid instead of enzymes. This is possible because the peptides of the invention are resistant to acid hydrolysis. In a preferred embodiment the expression construct also comprises a sequence coding for an affinity tag. This tag may be used for purifying the peptide. Preferably, the affinity tag is removed together with the linker and the N-terminal Methionine by cleaving the linker.

### Dosages

A cleaved mammal beta defensin, such as a human beta defensin, is preferably employed in pharmaceutical compositions in an amount which is effective to treat an inflammatory bowel disease, preferably with acceptable toxicity to the patient. For such treatment, the appropriate dosage will, of course, vary depending upon, for example, the chemical nature and the pharmacokinetic data of a compound of the present invention used, the individual host, the mode of administration and the nature and severity of the conditions being treated. However, in general, for satisfactory results in larger mammals, for example humans, an indicated daily dosage is preferably from about 0.0001 mg/kg body weight to about 10 mg/kg body weight, more preferably from about 0.001 mg/kg body weight to about 10 mg/kg body weight; such as 0.005 mg/kg body weight to 5 mg/kg body weight, more preferably from about 0.01 mg/kg body weight to about 10 mg/kg body weight, preferably from about 0.1 mg/kg body weight to about 10 mg/kg body weight, for example, administered in divided doses up to one, two, three, or four times a day. The compounds of preferred embodiments can be administered to larger mammals, for example humans, by similar modes of administration at similar dosages than conventionally used.

In one embodiment the daily dosage is preferably from 0.0001-10 mg/kg bodyweight, more preferably 0.001-10 mg/kg body weight, more preferably 0.005-5 mg/kg body weight.

In certain embodiments, the pharmaceutical compositions of preferred embodiments can include a cleaved mammal beta defensin, such as a cleaved human beta defensin, in an amount of about 0.5 mg or less to about 1500 mg or more per unit dosage form depending upon the route of administration, preferably from about 0.5, 0.6, 0.7, 0.8, or 0.9 mg to about 150, 200, 250, 300, 350, 400, 450, 500, 600, 700, 800, 900, or 1000 mg, and more preferably from about 1, 2, 3, 4, 5, 6, 7, 8, 9, 10, 15, 20, or 25 mg to about 30, 35, 40, 45, 50, 55, 60, 65, 70, 75, 80, 85, 90, 95, or 100 mg. In certain embodiments, however, lower or higher dosages than those mentioned above may be preferred. Appropriate concentrations and dosages can be readily determined by one skilled in the art.

In one embodiment, the cleaved mammalian beta defensin is administered at least once daily, such as at least twice daily, for example at least 3 times daily, such as at least 4 times daily.

The present invention is further described by the following examples that should not be construed as limiting the scope of the invention.

### EXAMPLES

### Example 1

### Purification of Cleaved Human Beta Defensin 2

Human Beta Defensin 2 (hBD2) hBD2 was produced recombinantly. A synthetic DNA fragment (DNA 2.0) encoding hBD2 was cloned into the pET-32(+) expression vector (Novagen). The resulting plasmid encoded a translational fusion peptide containing an N-terminal thioredoxin part followed by a his-tag, an enterokinase cleavage site and finally the hBD2 peptide. The expression plasmid was transformed into E. coli strain BL21.

An overnight culture of this strain was diluted 100 fold in TB-glycerol containing 100 μg/ml of ampicillin and grown to an OD600 of approximately 8 at 37° C. and induced with 0.5 mM of IPTG for 3 hours after which the cells were harvested by centrifugation. The his-tagged trx- hBD2 fusion peptide was purified on Ni-NTA beads (QIAGEN) using standard protocols. The his-tag purified fusion peptide was subsequently dialysed over-night into enterokinase buffer (50 mM tris-HCl pH 7.5, 1 mM CaCb) and cleaved with enterokinase to release mature hBD2. The hBD2 peptide was further purified by cation-exchange chromatography using Source 15 S matrix (Amersham Biosciences). The correct molecular weight of hBD2 was verified using MALDI-TOF mass spectrometry. The proper folding and disulphide-bridge topology of the hBD2 molecule was verified subsequently using tryptic digestion coupled with LC-MS and NMR spectroscopy.

Endotoxin was removed by preparative RP-HPLC at low pH, and the content of endotoxin was determined by a LAL assay (Endosafe KTA2) and the level was found to be below the detection limit of the assay (0.05 EU/mg). To ascertain that levels below the detection limit of the endotoxin assay were not able to stimulate PBMC, titration curves of stimulation with a very potent lipopolysaccharide (E. coli, O111:B4, Sigma L4391) were performed. Very low levels of this LPS (0.06 pg/ml) were able to stimulate PBMC to a detectable cytokine production.

Purified human beta defensin 2 formulated at approx. 100 mg/mL in PBS was diluted with 1 volume 400 mM Na-phosphate pH 2.3. pH was adjusted to 2.3 and subjected to acid hydrolysis for 16 hrs at 85° C. The cleaved human beta defensin 2 was subsequently purified by preparative RP-HPLC (15 μm C-18 substituted silica resin with a pore size of 100 Å) using a formic acid/ethanol based solvent system. The purified product was lyophilized and dissolved in PBS. The cleaved form of human beta defensin 2 was verified by LC-MS and the purity estimated by RP-UPLC to >99% at 280 nm and >96% at 215 nm.

### Example 2

In Vitro Analysis of Cleaved hBD2

### Isolation and Stimulation of PBMC.

Peripheral blood was drawn from healthy volunteers (with approval from the relevant ethical committee in Denmark). Heparinized blood was diluted 1/1 v/v with RPMI and were subjected to Ficoll density centrifugation within 2 h of drawing. Plasma was collected from the top from individual donors and was kept on ice until it was used at 2% in the culture medium (autologous culture medium). Isolated PBMC were resuspended in autologous culture medium and seeded in 96-well culture plates with 255.000 cells per well in a total of 200 μl. PBMC from the same donor were stimulated with 100, 10 or 1 pg/ml of hBD2 (either full-length or cleaved) either alone or together with LPS at 0.6 ng/ml or 20 ng/ml (E. coli, O111:B4, Sigma L4391), as a control on down-regulation of inflammatory cytokines 3.5 ng/ml Dexamethason (Sigma D8893) and 1 μg/ml Infliximab (Centocur B. V., NCI039843LA) was included. The concentrations of these compounds were based on initial titration studies, where the highest possible concentration without effect of viability was used. The supernatants were collected after incubation at 37° C. for 24 hours, and stored at −80° C.° until cytokine measurement. Viability was measured by Alamar Blue (Biosource, DALL 1100) to be sure that anti-inflammatory effects were not due to toxicity.

### Cytokine Measurements.

TNF and IL-10 production in supernatants was measured by flow cytometry with a human inflammation cytometric bead array (CBA) according to manufacturer's instructions (BDBioSciences) on a FACSarray flow cytometer (BDBiosciences).

### Data Analysis

All experiments were performed at least twice on several donors, with representative results shown. The data presented are expressed as mean plus/minus standard deviation (SD). Statistical significance was determined by 2-way ANOVA with the variables being treatment (hBD2, dexamethazone, etc.) and stimulation (LPS) followed by Bonferroni post-test as reported in the table legends. Differences were considered significant for p<0.05.

### Results

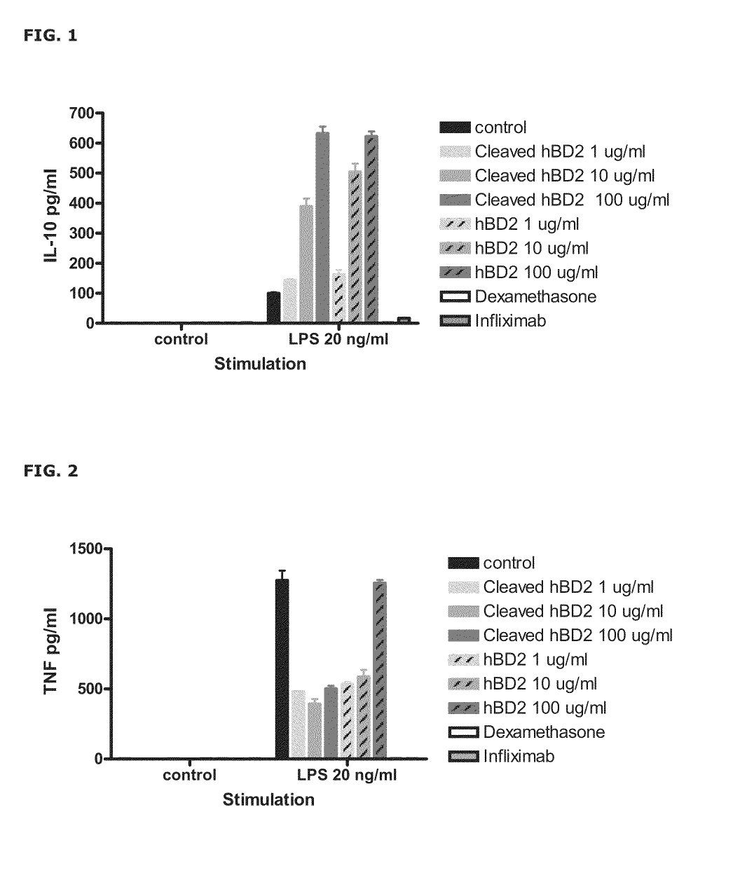

We have previously seen that full-length hBD2 exhibits anti-inflammatory effects by downregulation of TNF and upregulation of IL-10 when added to human PBMCs stimulated with LPS or another TLR ligand (WO 2010/007166). In FIGS. 1 and 2 in can be seen that cleaved hBD2 has the same anti-inflammatory activity in vitro as full-length hBD2 and these two peptides therefore potentially have the same anti-inflammatory activity in vivo. It is surprising and unexpected that the N-terminal 4 amino acids of the relatively short hBD2 peptide have no apparent effect on the bioactivity of the peptide. This is even more unexpected in view of the high degree of conservation of the four amino acids among humans and monkeys. In particular the Isoleucin at position 2 in full length beta-defensin 2 is fully conserved (FIG. 3). This normally indicates that the amino acid is important for the function of the peptide.

Cleaved hBD2 may therefore be used as an anti-inflammatory compound in autoimmune diseases or disorders. Such disorders (diseases) include inflammatory bowel disease (e.g., Crohn's Disease) and colitis (e.g., ulcerative colitis).

### Overview of Sequences

SEQ ID NO:1: human beta defensin 2 (hBD2)
SEQ ID NO:2: Rhesus macaque beta defensin 2
SEQ ID NO:3: Chimpanzee beta defensin 2
SEQ ID NO:4: Orangutan beta defensin 2
SEQ ID NO:5: Preferred cleaved human beta defensin 2
SEQ ID NO:6: Preferred cleaved rhesus macaque beta defensin 2
SEQ ID NO:7: Preferred cleaved chimpanzee beta defensin 2
SEQ ID NO:8: Preferred cleaved orangutan beta defensin 2
SEQ ID NO:9: Cleaved hBD2 variant (SEQ ID NO:5+1 N-terminal AA)
SEQ ID NO:10: Cleaved hBD2 variant (SEQ ID NO:5+2 N-terminal AA)
SEQ ID NO:11: Cleaved hBD2 variant (SEQ ID NO:5+3 N-terminal AA)

### SEQ ID NO:12: IGDPVT-

### SEQ ID NO:13: GDPVTC-

### SEQ ID NO:14: DPVTCL-

### SEQ ID NO:15: PVTCL-

### SEQ ID NO:16: PVTCLK-

SEQ ID NO:17: Consensus sequence 1
X₁ X₂ G A I C H P X₃ F C P R R Y K X₄ I G T C G L X₅ X₆ X₇ K C C K K P, wherein independently

### X₁ is K or R,

### X₂ is S or N,

### X₃ is V or G,

### X₄ is Q or H,

### X₅ is P or 5,

### X₆ is G or V, and

### X₇ is T or I.

SEQ ID NO:18: Consensus sequence 2
P V T C L X₁ X₂ G A I C H P X₃ F C P R R Y K X₄ I G T C G L X₅ X₆ X₇ K C C K K P Wherein independently;

### X₁ is K or R,

### X₂ is S or N,

### X₃ is V or G,

### X₄ is Q or H,

### X₅ is P or 5,

### X₆ is G or V, and

### X₇ is T or I.

SEQ ID NO:19: N-terminal truncation GIGD

It should be understood that for all numerical bounds describing some parameter in this application, such as “about,” “at least,” “less than,” and “more than,” the description also necessarily encompasses any range bounded by the recited values.

Accordingly, for example, the description at least 1, 2, 3, 4, or 5 also describes, inter alia, the ranges 1-2, 1-3, 1-4, 1-5, 2-3, 2-4, 2-5, 3-4, 3-5, and 4-5, et cetera.

For all patents, applications, or other reference cited herein, such as non-patent literature and reference sequence information, it should be understood that it is incorporated by reference in its entirety for all purposes as well as for the proposition that is recited. Where any conflict exits between a document incorporated by reference and the present application, this application will control. All information associated with reference gene sequences disclosed in this application, such as GeneIDs or accession numbers (typically referencing NCBI accession numbers), including, for example, genomic loci, genomic sequences, functional annotations, allelic variants, and reference mRNA (including, e.g., exon boundaries or response elements) and protein sequences (such as conserved domain structures) are hereby incorporated by reference in their entirety.

Headings used in this application are for convenience only and do not affect the interpretation of this application.

While this invention has been particularly shown and described with references to example embodiments thereof, it will be understood by those skilled in the art that various changes in form and details may be made therein without departing from the scope of the invention encompassed by the appended claims.

What is claimed is:

1. A peptide selected from the group consisting of:
a) a peptide having the amino acid sequence of any of SEQ ID NO: 5, 6, 7, 8, 9, 10, or 11;
b) an anti-inflammatory peptide variant of the peptide of a) having up to 1-5 amino acid substitutions relative to SEQ ID NO: 5, 6, 7, 8, 9, 10, or 11;
c) an anti-inflammatory peptide variant having an amino acid sequence with at least 80% sequence identity to any of SEQ ID NO: 5, 6, 7, or 8 and having an N-terminus selected from the group consisting of IGDPVT (SEQ ID NO:12), GDPVTC (SEQ ID NO:13), DPVTCL (SEQ ID NO:14) and PVTCL (SEQ ID NO:15);
d) an anti-inflammatory peptide having the consensus sequence: P V T C L X₁ X₂ G A I C H P X₃ F C P R R Y K X₄ I G T C G L X₅ X₆ X₇ K C C K K P, (SEQ ID NO:18) wherein independently
X₁ is K or R,
X₂ is S or N,
X₃ is V or G,
X₄ is Q or H,
X₅ is P or S,
X₆ is G or V, and
X₇ is T or I; and
e) an anti-inflammatory peptide having an amino acid sequence consisting of a sequence at least 80% identical to amino acids 2-41 of SEQ ID NO: 1, or a fragment thereof, wherein the fragment comprises a 37 amino acid polypeptide having an amino acid sequence at least 80% identical to SEQ ID NO: 5.

2. The peptide of claim 1, having the consensus sequence: P V T C L X₁ X₂ G A I C H P X₃ F C P R R Y K X₄ I G T C G L X₅ X₆ X₇ K C C K K P (SEQ ID NO:18), wherein independently
X₁ is K or R,
X₂ is S or N,
X₃ is V or G,
X₄ is Q or H,
X₅ is P or 5,
X₆ is G or V, and
X₇ is T or I.

3. The peptide of claim 1, having at least 85% sequence identity to any of SEQ ID NO:5, 6, 7, or 8.

4. The peptide of claim 1, being 37 amino acids long and having an amino acid sequence with up to 5 amino acid substitutions compared to SEQ ID NO:5.

5. The peptide of claim 1, being 37 amino acids long and having the amino acid sequence of SEQ ID NO:5.

6. A dimeric protein comprising the peptide of claim 1 as at least one monomer.

7. A composition comprising the peptide of claim 1 covalently linked to a chemical moiety selected from a heterologous peptide or polypeptide, an affinity tag, a carbohydrate, a lipid, or a synthetic polymer.

8. A method of manufacturing the peptide of claim 1, said method comprising recombinant expression, chemical synthesis, or acid cleavage of a full length mammalian beta-defensin 2.

9. The method of claim 8, wherein the recombinant expression is eukaryotic or prokaryotic.

10. The method of claim 8, comprising cleaving the N-terminal four amino acids from a mammalian beta-defensin 2 and subsequently purifying the cleaved peptide.

11. The method of claim 8, further comprising purification of the cleaved peptide and subsequent verification of the N-terminus, proper folding and/or correct presence of cysteine-bridges.

12. A polynucleotide comprising a nucleotide sequence selected from the group consisting of:
a) a polynucleotide coding for a polypeptide consisting of an N-terminal eukaryotic signal sequence linked to the peptide of claim 1; and
b) a polynucleotide coding for a polypeptide consisting of an N-terminal methionine linked to a cleavable linker, and the peptide of claim 1.

13. The polynucleotide of claim 12, further comprising an affinity tag, located between the N-terminal methionine and the cleavable linker.

14. The polynucleotide of claim 12, wherein the linker is cleavable by acid or by an enzyme.

15. An expression vector comprising a polynucleotide of claim 12.

16. A cell transduced or transfected with the expression vector of claim 15.

17. A pharmaceutical composition comprising the peptide of claim 1, and a pharmaceutical acceptable excipient, diluent or carrier.

18. A method of treatment of an inflammatory or autoimmune disorder, said method comprising administering to a subject in need thereof a peptide of claim 1, wherein the inflammatory disorder is selected from the group consisting of inflammatory bowel diseases (IBD), rheumatoid arthritis, psoriasis, osteoarthritis, multiple sclerosis, artherosclerosis, scleroderma (systemic sclerosis), lupus, systemic lupus erythematosus (SLE), (acute) glomerulonephritis, asthma, chronic obstructive pulmonary diseases (COPD), respiratory distress-syndrome (ARDS), vasculitis, uveitis, dermatitis, atopic dermatitis, alopecia, rhinitis (allergica), allergic conjunctivitis, myasthenia gravis, sclerodermitis, sarcoidosis, psoriatic arthritis, ankylosing spondylitis, juvenile idiopathic arthritis, Graves disease, Sjogren's syndrome, and Behget disease.

19. The method of claim 18, wherein the disorder is an inflammatory bowel disease or disorder.

20. The method of claim 18, wherein the disorder is rheumatoid arthritis.
